(19) United States
(12) Patent Application Publication
(10) Pub. No.: US 2019/0083609 A1
(43) Pub. Date: Mar. 21, 2019

(54) TOFACITINIB AS VACCINATION IMMUNE MODULATOR

(71) Applicants: HELMOLTZ ZENTRUM MÜNCHEN - DEUTSCHES FORSCHUNGSZENTRUM FÜR GESUNDHEIT AND UMWELT (GMBH), Neuherbert (DE); KLINIKUM RECHTS DER ISAR DER TECHNISCHEN UNIVERSITÄT MÜNCHEN, München (DE)

(72) Inventors: Jan GUTERMUTH, Affligem (BE); Carsten SCHMIDT-WEBER, München (DE); Markus OLLERT, Luxembourg (LU); Juan Antonio AGUILAR PIMENTEL, Griesstatt (DE); Fuchs HELMUT, Kolbermoor (DE); Valerie GAILUS-DURNER, Oberschleißheim (DE); Martin HRABE DE ANGELIS, Starnberg (DE)

(21) Appl. No.: 16/070,558

(22) PCT Filed: Jan. 18, 2017
(86) PCT No.: PCT/EP2017/050951

§ 371 Date: Jul. 17, 2018

(30) Foreign Application Priority Data
Jan. 18, 2016 (LU) 92950

(51) Int. Cl. A61K 39/35; A61K 31/506; A61K 31/435; A61K 39/00; A61P 37/08; A61K 39/39; A61K 31/519

(52) U.S. Cl. CPC A61K 39/35; A61K 31/506; A61K 31/435; A61K 39/0008; A61K 2039/55572; A61K 39/39; A61K 31/519; A61K 2039/577; A61P 37/08

(57) ABSTRACT

The invention relates to a pharmaceutical composition comprising Tofacitinib or a pharmaceutically acceptable salt thereof, and at least one antigen, allergen or autoallergen for use in medicine. Also encompassed is the use of said composition as a tolerogenic vaccine in the treatment or prevention of an immune disease. The invention further relates to Tofacitinib or a pharmaceutically acceptable salt thereof for use in tolerogenic vaccination. Another aspect of the invention is the use of Tofacitinib or a pharmaceutically acceptable salt thereof in the treatment or prevention of allergic rhinitis and/or autoimmune disease. Further the invention refers to a kit of parts comprising Tofacitinib or a pharmaceutically acceptable salt thereof, and at least one antigen or allergen.

The invention relates to a pharmaceutical composition comprising Tofacitinib or a pharmaceutically acceptable salt thereof, and at least one antigen or allergen for use in medicine. Also encompassed is the use of said composition as a tolerogenic vaccine in the treatment or prevention of an immune disease. The invention further relates to Tofacitinib or a pharmaceutically acceptable salt thereof for use in tolerogenic vaccination. Another aspect of the invention is the use of Tofacitinib or a pharmaceutically acceptable salt thereof in the treatment or prevention of allergic rhinitis and/or autoimmune diseases. Further the invention refers to a kit of parts comprising Tofacitinib or a pharmaceutically acceptable salt thereof, and at least one antigen or allergen.

Allergen-specific immunotherapy (ASIT) is the only curative therapy for allergies and reduces the symptoms by 30% in average, e.g. for hay fever. Further improvement of this tolerogenic vaccination is aiming on improvement closer to 100%, but also to extend the ASIT in atopic and more severe allergic patients, where ASIT is less efficient, e.g. in patients of the polysensitized category. The local inflammatory process appears to be a key problem, as it not only causes side effects, but also feeds back on the specific immune system and thus the immunologic memory (Akdis C A, Akdis M. Mechanisms of allergen-specific immunotherapy and immune tolerance to allergens, World Allergy Organ. J. 2015; 8:17). In particular, ASIT can lead to an initial boost of IgE production, which can lead to serious life threatening side effects such as an anaphylactic reaction, ranging from skin rash to cardiac or respiratory arrest and death. ASIT is also only available for the treatment of type 1 allergies, which are elicited by foreign proteins from pollen, food ect. In contrast, unwanted immune reactions against self-proteins from a patient, resulting in autoimmune diseases can to date not be treated with tolerogenic vaccination strategies.

The mechanisms underlying the induction of allergen specific immune tolerance are still largely unknown. However, the induction of allergen-specific IgG4 and a change of the T cell phenotype away from Th2 cells are assumed to underlie long-lasting treatment benefit (Chesne J, Schmidt-Weber C B, Esser von-Bieren J. The Use of Adjuvants for Enhancing Allergen Immunotherapy Efficacy, Immunol. Allergy Clin. North Am. 2016; 36:125-45, and Schmidt-Weber C B, Blaser K. Immunological mechanisms of specific allergen immunotherapy, Inflamm. Allergy Drug Targets 2006; 5:15-21). Regulatory T cells (Treg) were reported to be induced by ASIT (Wu Z. Antigen specific immunotherapy generates CD27(+) CD35(+) tolerogenic dendritic cells, Cell Immunol. 2013; 283:75-80, and Radulovic S, Jacobson M R, Durham S R, Nouri-Aria K T. Grass pollen immunotherapy induces Foxp3-expressing CD4+ CD25+ cells in the nasal mucosa, J. Allergy Clin. Immunol. 2008; 121:1467-72, 72 el) and may play a role in suppressing pro-allergic Th2 cells. It is currently unclear how the induction of regulatory T cells can be facilitated in context of antigen-specific vaccination, but it appears that allergic inflammation, specifically IL-4 can directly inhibit the induction of regulatory T cells by inhibiting the FOXP3 promoter. Similarly other pro- inflammatory mediators are counteracting Treg induction.

The use of immunosuppressant drugs may promote the induction of Tregs as long as the inhibition does not inhibit those signals that are necessary for T cell differentiation and also necessary for regulatory T cells. However previous attempts showed that Cyclosporine A inhibits the induction of Tregs and Corticosteroids are only minimally promoting Tregs. Further, it is known that JAK1/3 inhibitors are promoting Treg induction. However the role of these inhibitors and their collateral effects on T cells—interacting cell such as antigen presenting cells—was not investigated.

Tofacitinib is a known inhibitor of Janus Kinase 3, also known as JAK3. It is used as an immunosuppressive agent for organ transplants, xeno transplantation as well as for the treatment of autoimmune diseases such as psoriasis, Morbus Crohn and rheumatoid arthritis and other indications where immunosuppression is desirable (see for example U.S. Pat. No. 6,965,027 B2, U.S. Pat. No. 6,956,041 B2 and U.S. Pat. No. 7,091,208 B2). Tofacitinib is known to allow Treg proliferation, and it has been found that patients treated with Tofacitinib show a diminished responsiveness to 23-valent pneumococcal polysaccharide vaccine (PPSV-23), leading to low pneumococcal titers.

Currently are no immunomodulatory drugs available, that selectively maintain the desired antigen-vaccination effect, while blocking contra-tolerogenic inflammation. This effect would be desirable in any vaccination also against non-allergens (autoantigens). Therefore, a medical need exists for a tolerance inducing vaccination against (auto)antigens and (auto)allergens having high efficacy and reduced detrimental side effects.

The underlying problem of the present invention is the provision of a tolerance inducing vaccination against (auto)antigens and (auto)allergens, in which the aspired antigen-vaccination effect is maintained, while anaphylaxis and allergic inflammation are reduced.

The inventors of the present invention have conducted intensive studies and found surprisingly that a combination of Tofacitinib or a pharmaceutically acceptable salt thereof and an antigen or allergen significantly improves the efficacy of tolerance induction, while inhibiting unwanted and dangerous immune reactions. In particular, the unwanted T-helper cell Th1, Th2 and Th17-responses are inhibited, while the formation of tolerance-inducing regulatory T cells is not impaired. Also, the secretion of unwanted IgE-antibody levels is diminished. The improved control of local inflammation extends thereby the use of ASIT in more severe conditions such as polysensitized allergies as well as asthma and autoimmune diseases and potentially high risk patients suffering of mastocytosis or anaphylaxis.

In a first aspect the present invention relates to a pharmaceutical composition for use in medicine, wherein said composition comprises Tofacitinib or a pharmaceutically acceptable salt thereof, and at least one antigen or allergen.

As used herein and throughout the entire description, the term “pharmaceutical composition” relates to a composition which is suitable for administration to a patient, preferably a human patient. The particularly preferred pharmaceutical composition of this invention comprises Tofacitinib or a pharmaceutically acceptable salt thereof, and at least one antigen or allergen, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include liquid, frozen, and lyophilized compositions.

As used herein and throughout the entire description, “Tofacitinib” means 3-((3R, 4R)-4-methyl-3-[methyl-(7H-pyrrolo[2,3-d]pyrimidin-4-yl)-amino]-piperidin-1-yl)-3-oxopropionitrile. Tofacitinib has the chemical formula C₁₆H₂₀N₆O and the following structural formula:

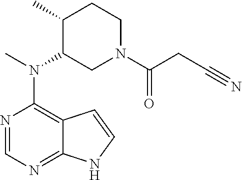

In general, the term “Tofacitinib” should be understood, unless otherwise indicated herein, to include any pharmaceutically acceptable form and salts of the compound. Tofacitinib may be present in a crystalline or amorphous form. Tofacitinib, salts of Tofacitinib, methods for synthesizing Tofacitinib, certain polymorphs of Tofacitinib, are disclosed in WO 01/42246, WO 02/096909, and WO 03/048162.

The present invention also relates to the pharmaceutically acceptable salts of Tofacitinib. Said salts are preferably acid addition salts of Tofacitinib. The acids which are used to prepare the pharmaceutically acceptable acid addition salts of Tofacitinib are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts. In some embodiments the pharmaceutically acceptable salt of Tofacitinib may be Tofacitinib citrate.

The term “pharmaceutically acceptable” may in particular mean approved by a regulatory agency or other generally recognized pharmacopoeia for use in animals, and more particularly in humans.

As used herein and throughout the entire description, the term “Antigen” means a B cell antigen or T cell antigen. In some embodiments, antigens may be proteins, polypeptides, peptides, lipoproteins, glycolipids, glycoproteins, polynucleotides, polysaccharides or are contained or expressed in cells. In some embodiments, antigens may be autoantigens. The autoantigen is preferably selected from a group consisting of DNA components, pancreatic islet antigens, GAD, insulinoma-associated antigen 2, insulin, zinc transporter, RPS, Alba-like, p62, CYPs, dUTPase, ASGPR2, SMA, matrix antigens, citrullin, carbamylated protein, liver and kidney microsomal antigens, Asialoglycoprotein receptor, neutrophil related antigens, extracellular matrix-proteins, MHC-II eluted peptides.

As used herein and throughout the entire description, the term “Autoantigen” means a normal (i.e. a body's own) protein or protein complex (and sometimes DNA or RNA) that is recognized by the immune system of patients suffering from a specific autoimmune disease. These antigens should not be, under normal conditions, the target of the immune system, but their associated T cells are not deleted and instead attack (or support B cells), which leads to an autoimmune disease.

As used herein and throughout the entire description, the term “Allergen” means any substance that can cause an undesired (e.g., a Type 1 hypersensitive) immune response (i.e., an allergic response or reaction) in a subject. Allergens include plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der PI antigen) and cockroach allergens. Drug allergens include allergens that are or are associated with antibiotics, NSAIDs, anesthetics, etc. Pollen allergens include grass allergens, tree allergens, weed allergens, flower allergens, etc. In some embodiments the allergen is a plant allergen, animal allergen, insect sting allergen, insect allergen, cosmetic allergen, drug allergen and/or food allergen. In some embodiments the allergen is a plant allergen, animal allergen, insect sting allergen, insect allergen, and/or food allergen. In some embodiments the allergen is a food allergen. In some embodiments the allergen is an animal allergen. The term allergen also includes an autoallergen. In some embodiments the allergen may be an autoallergen.

In some embodiments, the pharmaceutical composition further comprises at least one adjuvant. The adjuvant stimulates the immune system's response to the antigen or allergen. Exemplary adjuvants for use in accordance with the present invention include inorganic compounds such as alum, aluminum hydroxide, aluminum phosphate, calcium phosphate hydroxide, mineral oils, such as paraffin oil, virosomes, bacterial products, such as killed bacteria Bordetella pertussis,

Mycobacterium bovis, toxoids, nonbacterial organics, such as squalene, thimerosal, detergents (Quil A), cytokines, such as IL-1, IL-2, IL-10 and IL-12, monophosphoryl lipid A (MPL) and complex compositions such as Freund's complete adjuvant, and Freund's incomplete adjuvant. Generally, the adjuvant used in accordance with the present invention preferably potentiates the immune response to the antigen or allergen of the invention and/or modulates it towards the desired immune responses. In a preferred embodiment the adjuvant is selected from monophosphoryl lipid A (MPL), alum or other activators of antigen presenting cells. In some embodiments the adjuvant may be selected from monophosphoryl lipid A (MPL), alum or other activators of dendritic cells. In some embodiments the adjuvant may be alum or monophosphoryl lipid A (MPL). In some embodiments the adjuvant may be alum. In some embodiments the adjuvant may be monophosphoryl lipid A (MPL). In some embodiments the adjuvant may be a combination of at least 2 adjuvants, such as of the adjuvants described above. In some embodiments the adjuvant may be a combination of 2, 3, 4, 5, 6, 7, 8, 9, 10 or more adjuvants. In some embodiments the adjuvant may be a combination of alum and monophosphoryl lipid A (MPL). In some embodiments, the pharmaceutical composition further comprises at least one pharmaceutically acceptable carrier. In some embodiments Tofacitinib or a pharmaceutically acceptable salt thereof may be included in a pharmaceutically acceptable carrier that also contains the antigen or allergen. In some embodiments Tofacitinib or a pharmaceutically acceptable salt thereof may be included in a pharmaceutically acceptable carrier that also contains the antigen or allergen and at least one adjuvant and/or further excipient.

As used herein and throughout the entire description, the terms “carrier” and “excipient” are used interchangeably herein. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc), humectants (e.g. propylene, glycol, glycerol, sorbitol). The person skilled in the art will readily be able to choose suitable pharmaceutically acceptable carriers or excipients, depending, e.g., on the formulation and administration route of the pharmaceutical composition.

A non-exhaustive list of exemplary pharmaceutically acceptable carriers or excipients includes (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologie, 5^{th} Ed., Govi-Verlag Frankfurt (1997).

The pharmaceutically acceptable carrier is preferably a hydrogel, containing Tofacitinib or a pharmaceutically acceptable salt thereof and the antigen or allergen. In another preferred embodiment said pharmaceutically acceptable carrier further contains at least one adjuvant and/or further excipient.

The pharmaceutical composition of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include
topical routes (such as epicutaneous, inhalational, nasal, opthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

In some embodiments the administration may be a topical route, in particular epicutaneous or subcutaneous. In some embodiments the administration may be a parental route, in particular intramuscular or subcutaneous, preferably intramuscular.

Another aspect of the present invention is a pharmaceutical composition for use as a tolerogenic vaccine in the treatment or prevention of an immune disease, wherein said composition comprises Tofacitinib or a pharmaceutically acceptable salt thereof and at least one antigen or allergen.

As used herein and throughout the entire description, the term “tolerogenic vaccine” means a vaccine that induces immunological tolerance to alleviate the inflammatory autoimmune diseases as well as other inflammatory diseases of metabolism, neurodegeneration, allergic hypersensitivity, and transplantation rejection. Preferably the tolerogenic vaccine induces long-term, antigen or allergen-specific, inhibitory memory that blocks pathogenic T cell responses via loss of effector T cells and gain of regulatory T cell function.

In some embodiments, the pharmaceutical composition further comprises at least one adjuvant.

In some embodiments, the pharmaceutical composition further comprises at least one pharmaceutically acceptable carrier.

In some embodiments, the immune disease may be an allergy or asthma.

As used herein and throughout the entire description, the term “Allergy” is any condition where there is an undesired (e.g., a Type 1 hypersensitive) immune response (i.e., allergic response or reaction) to a substance. Such substances are referred to herein as allergens. Allergies or allergic conditions include allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. The allergic reaction may be the result of an immune reaction to any allergen. In some embodiments, the allergy is a food allergy. Food allergies include milk allergies, egg allergies, nut allergies, fish allergies, shellfish allergies, soy allergies or wheat allergies. The allergy is preferably selected from allergic rhinitis, hay fever, food allergy and allergic airway inflammation. The allergy is more preferably allergic rhinitis. The allergy is more preferably allergic airway inflammation. The allergy is more preferably food allergy.

As used herein and throughout the entire description, the term “Asthma” is a chronic inflammatory disease of the airways characterized by variable and recurring symptoms, reversible airflow obstruction and bronchospasm, as a result to an undesired (e.g., a Type 1 hypersensitive) immune response (i.e., allergic response or reaction) to an allergen. The asthma is preferably selected from allergic asthma, or non-atopic asthma with uncertain trigger (e.g. Autoimmune triggered or autoallergies). In some embodiments the asthmas may be selected from allergic asthma, or asthma with unknown trigger. In some embodiments the asthma may be allergic asthma. In some embodiments the asthma may be non-atopic asthma with uncertain or unknown trigger. In some embodiments the non-topic asthma may be autoimmune triggered. In some embodiments the non-topic asthma may be autoallergie triggered.

In some embodiments, the immune disease may be an autoimmune disease. The autoimmune disease is preferably selected from Type I diabetes, multiple sclerosis, ulcerative colitis, Crohn's disease, lupus erythematosus, psoriasis, rheumatoid arthritis, psoriatic arthritis, GravesDisease, Hashimoto's Thyroiditis, Vitiligo, Pernicious Anemia, Scleroderma, Glomerulonephritis, Sjogren's Syndrome, Bullous pemphigoid and Pemhigus vulgaris. In some embodiments the autoimmune disease may be Type I diabetes. In some embodiments the autoimmune disease may be multiple sclerosis. In some embodiments the autoimmune disease may be rheumatoid arthritis. In some embodiments the autoimmune disease may be psoriasis. In some embodiments the autoimmune disease may be lupus erythematosus. In some embodiments the autoimmune disease may be an inflammatory bowel disease. In some embodiments the autoimmune disease may be GravesDisease. In some embodiments the autoimmune disease may be Hashimoto's Thyroiditis.

In some embodiments the autoimmune disease may be Vitiligo. In some embodiments the autoimmune disease may be Pernicious Anemia. In some embodiments the autoimmune disease may be Glomerulonephritis. In some embodiments the autoimmune disease may be ulcerative colitis. In some embodiments the autoimmune disease may be Sjogren's Syndrome. The disease is preferably Type I diabetes and/or multiple sclerosis, GravesDisease, Hashimoto's Thyroiditis, Vitiligo, Pernicious Anemia, Glomerulonephritis, ulcerative colitis, Crohn's disease, Sjogren's Syndrome, Bullous pemphigoid, Scleroderma and Pemphigus vulgaris. The disease is more preferably ulcerative colitis, Crohn's disease, Pemphigus vulgaris, Bullous Pemphigoid and Scleroderma. The disease is more preferably ulcerative colitis and/or Crohn's disease, Pemphigus vulgaris, Bullous Pemphigoid and Scleroderma.

In some embodiments the composition is administered to a subject in need thereof in an amount effective to treat or prevent said immune diseases. The subject is preferably a human subject.

As used herein and throughout the entire description, the term “Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

As used herein and throughout the entire description, the term “Amount effective” in the context of a composition or dosage form for administration to a subject refers to an amount of the composition or dosage form that produces one or more desired immune responses in the subject, in particular, the generation of a tolerogenic immune response. Therefore, in some embodiments, an amount effective is any amount of a composition provided herein that produces one or more of these desired immune responses. This amount can be for in vitro or in vivo purposes. For in vivo purposes, the amount can be one that a clinician would believe may have a clinical benefit for a subject in need of antigen-specific tolerization. Such subjects include those that have or are at risk of having an immune disease, in particular an allergy, asthma and/or autoimmune disease.

Amounts effective can involve only reducing the level of an undesired immune response, although in some embodiments, it involves preventing an undesired immune response altogether. Amounts effective can also involve delaying the occurrence of an undesired immune response.

An amount that is effective can also be an amount of a composition provided herein that produces a desired therapeutic endpoint or a desired therapeutic result. Amounts effective, preferably, result in a tolerogenic immune response in a subject to an antigen and/or allergen. The achievement of any of the foregoing can be monitored by routine methods.

In some embodiments of any of the compositions provided, the amount effective is one in which the desired immune response persists in the subject for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer. In other embodiments of any of the compositions provided, the amount effective is one which produces a measurable desired immune response, for example, a measurable decrease in an immune response (e.g., to a specific antigen), for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

Another aspect of the present invention is Tofacitinib or a pharmaceutically acceptable salt thereof for use in tolerogenic vaccination. In some embodiments Tofacitinib or a pharmaceutically acceptable salt thereof may be supports a tolerogenic vaccination. In some embodiments Tofacitinib or a pharmaceutically acceptable salt thereof may be administered orally shortly before or simultaneously with an antigen or allergen vaccination. In some embodiments Tofacitinib or a pharmaceutically acceptable salt thereof may be administered orally shortly before, during and shortly after or simultaneously with an antigen or allergen vaccination. In some embodiments Tofacitinib or a pharmaceutically acceptable salt thereof may be administered orally shortly before, during and shortly after an antigen or allergen vaccination. In some embodiments Tofacitinib or a pharmaceutically acceptable salt thereof may be administered orally shortly before an antigen or allergen vaccination. In some embodiments Tofacitinib or a pharmaceutically acceptable salt thereof may be administered simultaneously with an antigen or allergen vaccination. In some embodiments the used vaccine contains further at least one adjuvant. In some embodiments the used vaccine contains further at least one excipient. In some embodiments Tofacitinib or a pharmaceutically acceptable salt thereof may be an immune modulator, in particular a tolerance inducer. In some embodiments Tofacitinib or a pharmaceutically acceptable salt thereof may be promotes the induction of the allergen or antigen tolerance.

As used herein and throughout the entire description, the term “tolerogenic vaccination” means a vaccination that re-establishes immunological tolerance, restores immune homeostasis, and thereby reverses an immune disease. The immune disease may be in some embodiments an allergy, allergic asthma, autoallergy or autoimmune disease. Tolerogenic vaccination is achieved by a tolerogenic immune response.

As used herein and throughout the entire description, the term “support of a tolerogenic vaccination” means any action of a compound or composition that facilitates or helps to achieve a tolerogenic immune response.

As used herein and throughout the entire description, the term “Tolerogenic immune response” means any immune response that can lead to immune suppression specific to an allergen, antigen or a cell, tissue, organ, etc. that expresses such an antigen. Such immune responses include any reduction, delay or inhibition in an undesired immune response specific to the antigen or cell, tissue, organ, etc. that expresses such antigen. Such immune responses also include any stimulation, production, induction, promotion or recruitment in a desired immune response specific to the allergen, antigen or cell, tissue, organ, etc. that expresses such antigen.

Tolerogenic immune responses, therefore, include the absence of or reduction in an undesired immune response to an antigen that can be mediated by antigen reactive cells as well as the presence or promotion of suppressive cells.

Tolerogenic immune responses as provided herein include immunological tolerance. To “generate a tolerogenic immune response” refers to the generation of any of the foregoing immune responses specific to an antigen or cell, tissue, organ, etc. that expresses such antigen. The tolerogenic immune response can be the result of MHC Class I-restricted presentation and/or MHC Class II-restricted presentation and/or B cell presentation, etc.

Tolerogenic immune responses include any reduction, delay or inhibition in CD4+ T cell, CD8+ T cell or B cell proliferation and/or activity. Tolerogenic immune responses also include a reduction in antigen- specific antibody production. Tolerogenic immune responses can also include any response that leads to the stimulation, induction, production or recruitment of regulatory cells, such as CD4+ Treg cells, CD8+ Treg cells, Breg cells, etc. In some embodiments the tolerogenic immune response, is one that results in the conversion to a regulatory phenotype characterized by the production, induction, stimulation or recruitment of regulatory cells.

Tolerogenic immune responses also include any response that leads to the stimulation, production or recruitment of CD4+ Treg cells and/or CD8+ Treg cells. CD4+ Treg cells can express the transcription factor FoxP3 and inhibit inflammatory responses and auto-immune inflammatory diseases (Human regulatory T cells in autoimmune diseases. Cvetanovich GL, Hafler D A. Curr Opin Immunol. 2010 December; 22(6):753-60. Regulatory T cells and autoimmunity, Vila J, Isaacs J D, Anderson A E. Curr Opin Hematol. 2009 July; 16(4):274-9). Such cells also suppress T-cell help to B-cells and induce tolerance to both self and foreign antigens (Therapeutic approaches to allergy and autoimmunity based on FoxP3+regulatory T-cell activation and expansion. Miyara M, Wing K, Sakaguchi S. J Allergy Clin Immunol. 2009 April; 123(4):749-55). CD4+ Treg cells recognize antigen when presented by Class II proteins on APCs. CD8+Treg cells, which recognize antigen presented by Class I (and Qa- 1), can also suppress T-cell help to B-cells and result in activation of antigen- specific suppression inducing tolerance to both self and foreign antigens. Disruption of the interaction of Qa-1 with CD8+Treg cells has been shown to dysregulate immune responses and results in the development of auto-antibody formation and an auto-immune lethal systemic-lupus-erythematosus (Kim et al., Nature. 2010 Sep. 16, 467 (7313): 328-32). CD8+Treg cells have also been shown to inhibit models of autoimmune inflammatory diseases including rheumatoid arthritis and colitis (CD4+CD25+regulatory T cells in autoimmune arthritis. Oh S, Rankin A L, Caton A J. Immunol Rev. 2010 January; 233(I):97-111, Regulatory T cells in inflammatory bowel disease. Boden E K, Snapper S B. Curr Opin Gastroenterol. 2008 November; 24(6):733-41). In some embodiments, the compositions provided can effectively result in both types of responses (CD4+ Treg and CD8+ Treg). In other embodiments, FoxP3 can be induced in other immune cells, such as macrophages, iNKT cells, etc., the compositions provided herein can result in one or more of these responses as well.

Tolerogenic immune responses may be also include in some embodiments the induction of regulatory cytokines, such as Treg cytokines; induction of inhibitory cytokines; the inhibition of inflammatory cytokines (e.g., IL-4, IL-Ibeta, IL-5, TNF-alpha, IL-6, GM-CSF, IFN-γ, IL-2, IL-9, IL-12, IL-17, IL-18, IL-21, IL-22, IL-23, M-CSF, C reactive protein, acute phase protein, chemokines (e.g., MCP-1, RANTES, MIP-1 alpha, MIP-1 beta, MIG, ITAC or IP-10), the production of anti-inflammatory cytokines (e.g., IL-4, IL-13, IL-10, etc.), chemokines (e.g., CCL-2, CXCL8), proteases (e.g., MMP-3, MMP-9), leukotrienes (e.g., CysLT-1, CysLT-2), prostaglandins (e.g., PGE2) or histamines; the inhibition of polarization to a Th17, Th1 or Th2 immune response; the inhibition of effector cell-specific cytokines: Th17 (e.g., IL-17, IL-25), Th1 (IFN-gamma), Th2 (e.g., IL-4, IL-13); the inhibition of Th1-, Th2- or Th17-specific transcription factors; the inhibition of proliferation of effector T cells; the induction of apoptosis of effector T cells; the induction of tolerogenic dendritic cell-specific genes; the induction of FoxP3 expression; the inhibition of IgE induction or IgE-mediated immune responses; the inhibition of antibody responses (e.g., antigen-specific antibody production); the inhibition of T helper cell response; the production of TGF-beta and/or IL-10; the inhibition of effector function of autoantibodies (e.g., inhibition in the depletion of cells, cell or tissue damage or complement activation); etc. In some embodiments, the tolerogenic immune response includes the production of anti-inflammatory cytokines (e.g., IL-4 and/or IL-10). In some embodiments, the tolerogenic immune response is the reduction of antigen- specific antibodies and/or CD4+ T helper cells and/or B cells. Assessing CD4+ T helper cell or B cell stimulation may include analyzing CD4+ T helper cell or B cell number, phenotype, activation and/or cytokine production.

Any of the foregoing may be measured in vivo in one or more animal models or may be measured in vitro. One of ordinary skill in the art is familiar with such in vivo or in vitro measurements. Undesired immune responses or tolerogenic immune responses can be monitored using, for example, methods of assessing immune cell number and/or function, tetramer analysis, ELISPOT, flow cytometry-based analysis of cytokine expression, cytokine secretion, cytokine expression profiling, gene expression profiling, protein expression profiling, analysis of cell surface markers, PCR-based detection of immune cell receptor gene usage (see T. Clay et al., “Assays for Monitoring Cellular Immune Response to Active Immunotherapy of Cancer” Clinical Cancer Research 7: 1127-1135 (2001)), etc. Undesired immune responses or tolerogenic immune responses may also be monitored using, for example, methods of assessing protein levels in plasma or serum, T cell or B cell proliferation and functional assays, etc. In some embodiments, tolerogenic immune responses can be monitored by assessing the induction of FoxP3.

Preferably, tolerogenic immune responses lead to the inhibition of the development, progression or pathology of the diseases, disorders or conditions described herein. Whether or not the inventive compositions can lead to the inhibition of the development, progression or pathology of the diseases, disorders or conditions described herein can be measured with animal models of such diseases, disorders or conditions. In some embodiments, the reduction of an undesired immune response or generation of a tolerogenic immune response may be assessed by determining clinical endpoints, clinical efficacy, clinical symptoms, disease biomarkers and/or clinical scores. Undesired immune responses or tolerogenic immune responses can also be assessed with diagnostic tests to assess the presence or absence of a disease, disorder or condition as provided herein. Undesired immune responses can further be assessed by methods of measuring proteins levels and/or function in a subject. In embodiments, methods for monitoring or assessing undesired allergic responses include assessing an allergic response in a subject by skin reactivity and/or allergen-specific antibody production.

Another aspect of the present invention is Tofacitinib or a pharmaceutically acceptable salt thereof for use in the treatment or prevention of allergic rhinitis. In some embodiments Tofacitinib or a pharmaceutically acceptable salt thereof may be used in the treatment of allergic rhinitis. In some embodiments Tofacitinib or a pharmaceutically acceptable salt thereof may be used in the prevention of allergic rhinitis. In some embodiments Tofacitinib or a pharmaceutically acceptable salt thereof may be administered orally shortly before or simultaneously with an allergen vaccination for allergic rhinitis. In some embodiments Tofacitinib or a pharmaceutically acceptable salt thereof may be administered orally shortly before, during and/or shortly after or simultaneously with an allergen vaccination for allergic rhinitis. In some embodiments Tofacitinib or a pharmaceutically acceptable salt thereof may be administered orally shortly before, during and/or shortly after an allergen vaccination for allergic rhinitis. In some embodiments Tofacitinib or a pharmaceutically acceptable salt thereof may be administered orally shortly before an allergen vaccination for allergic rhinitis. In some embodiments Tofacitinib or a pharmaceutically acceptable salt thereof may be administered simultaneously with an allergen vaccination for allergic rhinitis.

Another aspect of the present invention is Tofacitinib or a pharmaceutically acceptable salt thereof for use in the treatment or prevention of an immune disease, wherein said compound is administered orally shortly before or simultaneously with an antigen vaccination. In some embodiments the immune disease may be an autoimmune disease and the antigen an autoantigen. In some embodiments Tofacitinib or a pharmaceutically acceptable salt thereof may be administered orally shortly before, during and shortly after or simultaneously with an antigen vaccination. In some embodiments Tofacitinib or a pharmaceutically acceptable salt thereof may be administered orally shortly before, during and/or shortly after an antigen and/or autoantigen vaccination. In some embodiments Tofacitinib or a pharmaceutically acceptable salt thereof may be administered orally shortly before an antigen and/or autoantigen vaccination. In some embodiments Tofacitinib or a pharmaceutically acceptable salt thereof may be administered simultaneously with an antigen and/or autoantigen vaccination.

Another aspect of the present invention is a kit of parts comprising Tofacitinib or a pharmaceutically acceptable salt thereof, and at least one antigen or allergen. In some embodiments the kit of parts comprises Tofacitinib or a pharmaceutically acceptable salt thereof, and at least one antigen. In some embodiments the kit of parts comprises Tofacitinib or a pharmaceutically acceptable salt thereof, and at least one allergen. In some embodiments the kit of parts may further comprises at least one adjuvant and/or pharmaceutically acceptable carrier. In some embodiments Tofacitinib or a pharmaceutically acceptable salt thereof and the allergen or antigen may be included in a pharmaceutically acceptable carrier. In some embodiments Tofacitinib or a pharmaceutically acceptable salt thereof, the allergen or antigen and an adjuvant may be included in a pharmaceutically acceptable carrier. In some embodiments Tofacitinib or a pharmaceutically acceptable salt thereof and the allergen or antigen may be included in a pharmaceutically acceptable carrier, with at least one adjuvant and/or further excipient.

The present invention also envisions a method of tolerogenic vaccination of a subject in need thereof, comprising administering to said subject an efficient amount of Tofacitinib or a pharmaceutically acceptable salt thereof. Said method of tolerogenic vaccination preferably comprises further administering at least one antigen or allergen. The above described aspects, embodiments, definitions, etc. are also applicable to said method of treatment, mutatis mutandis.

### BRIEF DESCRIPTION OF THE DRAWINGS

FIG. 1: Controls and treatment protocol. Four groups of mice each group consisting of 14 mice were run in parallel. First Group (Control): animals have been exposed to OVA aerosol only. Second Group (OVA-Allergic): animals have been sensitized with OVA and exposed to OVA aerosol. Third Group (OVA-ASIT): animals have been sensitized with OVA, treated with SIT (OVA) and exposed to OVA aerosol. Fourth Group (OVA-ASIT-Tofa): animals have been sensitized with OVA, treated with SIT (OVA) during a Tofacitinib oral treatment and afterwards exposed to OVA aerosol. OVA denotes Ovalbumin. SIT denotes specific immunotherapy. Intraperitoneal is denoted by i.p. Subcutaneous is denoted by s.c.

FIG. 2: Schematic representation of the levels of allergic symptoms of the different mouse groups, the intensity of the disease progression is referring to the allertic airway inflammation parameters scored suchs as Immunoglobulins, cytokines, cell infiltration .ASIT: allergen-specific-immunotherapy, OVA: ovalbumin, Tofa: Tofacitinib.

FIG. 3: Under cell culture conditions T cells were stimulated by two different doses of activating stimulant. Tofacitinib significantly decreased proliferation of T cells in a dose-dependent manner. Tofa denotes Tofacitinib. DMSO denotes dimethyl sulfoxide.

FIG. 4: Under cell culture conditions B cells were stimulated by two different doses of activating stimulant. A) Tofacitinib significantly decreased proliferation of B cells. B) The treatment with Tofancitinib decreased the IgE production by B cells under cell culture conditions.

FIG. 5: Analysis of plasma levels of immunoglobulin isotope IgE after challenge. The serum levels of total IgE decreased in the OVA-ASIT groups in comparison with the allergic group. This effect was even more pronounced when OVA-ASIT was combined with the oral administration of Tofanitinib. ASIT: allergen-specific-immunotherapy, OVA: ovalbumin, Tofa: Tofacitinib.

FIG. 6: Analysis of plasma levels of immunoglobulin isotopes IgG, after challenge. The levels of total IgG1 increased during ASIT. ASIT: allergen-specific-immunotherapy, OVA: ovalbumin, Tofa: Tofacitinib.

FIG. 7: Systemic (plasma) cytokine profiles in plasma showed a clear reduction of the levels of IL-6, 1L-1beta and 11-2 in the OVA-ASIT group. This effect was even more pronounced in the OVA-ASIT-Tofa group. ASIT: allergen-specific-immunotherapy, OVA: ovalbumin, Tofa: Tofacitinib.

FIG. 8: Allergic response in the bronchoalveolar lavage (BAL). BAL cell infiltration into the lung was determined by flow cytometry analysis. Cell counts in the BAL showed a significant reduction of infiltrating cells in the lungs of mice treated with SIT. This effect was significantly more pronounced in mice which were additionally treated with Tofacitinib during ASIT. Moreover, the total number as well as the relative percentage of eosinophils was significantly decreased, confirming the protective effect of Tofacitinib. ASIT: allergen-specific-immunotherapy, OVA: ovalbumin, Tofa: Tofacitinib.

FIG. 9: Cytokine profile in the broncho alveolar lavage fluid (BAL). It shows a significant reduction of most of the cytokines in the OVA-ASIT groups which is tendentially even more pronounced in the OVA-ASIT-Tofa group, confirming the regulatory effect of Tofacitinib in BAL. ASIT: allergen-specific-immunotherapy, OVA: ovalbumin, Tofa: Tofacitinib.

FIG. 10: Effect of Tofacitinib on the differentiation of human FOXP3+ CD4+ T cells. Naive CD4+ T cells of human blood donors (n=3) were stimulated with anti-CD3/anti-CD28, IL-2 and TGF-β1 to induce FOXP3 expression. In addition to this polarization control, Tofacitinib, Rapamycin or Cycloysporine A were added to the medium in different concentrations. (A) Flow cytometry plots of anti-CD4 and anti-FOXP3 staining are shown for one representative donor. (B) The percentages of FOXP3+cells within the living CD4+ T cell population under control, Tofacitinib, Rapamycin and Cyclosporine A condition is given for the three donors. Bars indicate the median. On average 0.1 μM and 1 μM Tofacitinib were able to increase the percentage of FOXP3 expressing cells about 2.4 (+/−0.7)-2.5 (+/−1.3)-fold. Rapamycin treatment did not inhibit FOXP3 expression and resulted in 1.3-fold higher expression. The lowest concentration of Cyclosporine A (0.003 μM) showed no effect on FOXP3 induction, whereas the two higher concentrations (0.033 μM, 0.333 μM) reduced the percentage of FOXP3⁺ cells within the CD4⁺ T cell compartment.

Further, the invention shall be explained in more detail by the following Examples.

### EXAMPLES

1) Methods and Materials

1.1) Reagents

Tofacitinib citrate (CP-690550) (Selleckchem-Biozol Diagnostica Vertrieb GmbH, Eching, Germany) was dissolved to 50 mg/ml in a sterile solution of DMSO, and administrated by oral gavage feeding in 10.8 mg/kg per dose in a total volume of 200 μl water.

1.2) Animals

Female C57BL/6j mice were obtained from The Jackson Laboratory, Germany. All mice were housed under specific pathogen free conditions in accordance to FELASA guidelines. Mouse husbandry and all animal experiments were carried in accordance with German legal guidelines and following the approval (approval number 5 55.2-1-54-2532-30-14) of the responsible animal welfare authorities and the ethics board of the district government of Upper Bavaria, Germany (full name: Regierung von Oberbayern, Sachgebiet 54).

1.3) Model of Induction of Allergic Airway Inflammation and Allergen-Specific Immunotherapy

For allergic sensitization and allergen-challenge, mice were treated twice by intraperitoneal injection of 10 pg OVA (chicken ovalbumin, Sigma, Germany) and 0.5 mg aluminum hydroxide (alum; 40 mg magnesium/40 mg alum per ml; inject® ThermoFischer, Rockford, USA) in 200 μl phosphate buffered saline (PBS) at day (D) 1 and D-7 as previously described (Aguilar-Pimentel J A, Alessandrini F, Huster K M, Jakob T, Schulz H, Behrendt H, et al. Specific CD8 T cells in IgE-mediated allergy correlate with allergen dose and allergic phenotype, Am J Respir Crit Care Med 2010; 181:7-16, and Fuchs H, Gailus-Durner V, Adler T, Aguilar-Pimentel J A, Becker L, Calzada-Wack J, et al. Mouse phenotyping, Methods 2011; 53:120-35). Subsequently, mice were challenged by inhalative exposure to OVA aerosol (1% in PBS) for 10 min once a day at D-49, 52 and 55. 24 h after the last challenge (D-56) blood samples were collected and animals sacrificed to obtain bronchoalveolar lavage (BAL) samples.

For allergen-specific-immunotherapy (ASIT), animals were treated by subcutaneous injection of 1 mg OVA D-22, and 0.5 mg OVA D-29. For treatment with Tofacitinib, two doses per day were administrated on D-20 to D-24, and D-27 to D-31.

1.4) Controls and Treatment Protocol:

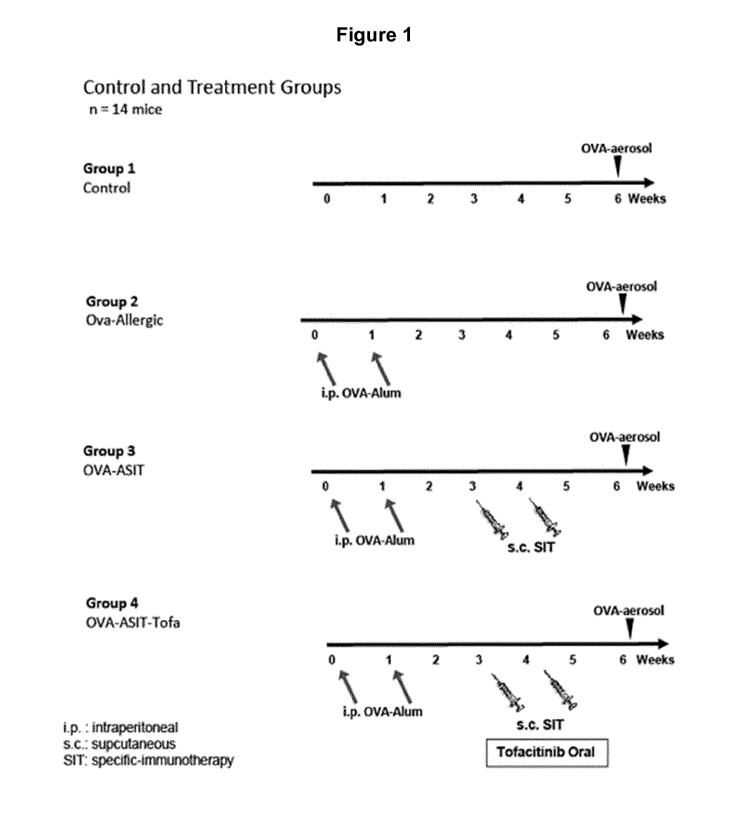

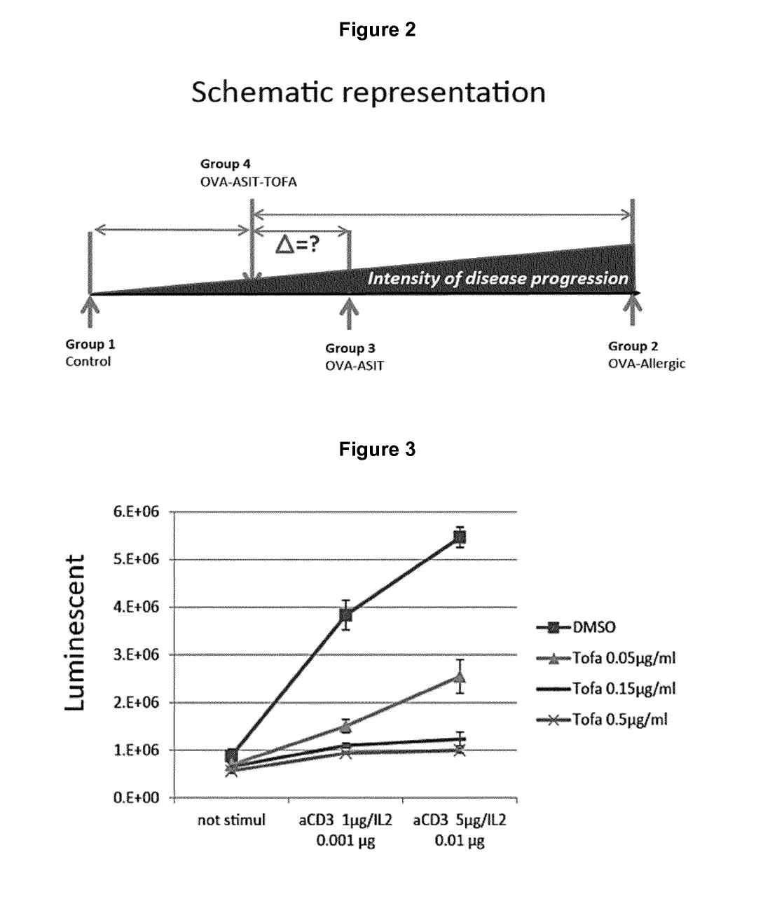

Four experimental groups of mice each group consisting of 14 mice were analyzed in parallel (FIGS. 1 and 2):

Controls: Non-sensitized mice, exposed to OVA-aerosol only.

OVA-Allergic: Mice sensitized with OVA and exposed to OVA-aerosol (challenge).

OVA Allergic +ASIT (OVA-ASIT): Mice sensitized with OVA/alum, treated with OVA-ASIT and challenged with OVA aerosol.

OVA Allergic +ASIT-Tofacitinib (OVA-TOFA-ASIT): Mice sensitized with OVA, treated with OVA-ASIT with concomitant gastric Tofacitinib feeding and subsequent exposure to OVA-aerosol.

1.5) Determination of Allergic Responses in Plasma

Mouse blood samples were collected before and after challenge as previously described (Aguilar-Pimentel J A, Alessandrini F, Huster K M, Jakob T, Schulz H, Behrendt H, et al. Specific CD8 T cells in IgE-mediated allergy correlate with allergen dose and allergic phenotype, Am J Respir Crit Care Med 2010; 181:7-16; Fuchs H, Gailus-Durner V, Adler T, Aguilar-Pimentel J A, Becker L, Calzada-Wack J, et al. Mouse phenotyping, Methods 2011; 53:120-35 and Fulton R J, McDade R L, Smith P L, Kienker L J, Kettman J R, Jr. Advanced multiplexed analysis with the FlowMetrix system, Clin Chem 1997; 43:1749-56) by puncturing the retro-orbital plexus (Li-heparin-coated tubes, KADE, Numbrecht, Germany) under isoflurane anesthesia. Blood samples were centrifuged (10 min, 5000×g) in a refrigerated centrifuge to separate cells and plasma. Plasma total IgE was measured using a classical immunoassay isotope-specific sandwich ELISA. In brief, plasma samples and standards for murine IgE (Mouse IgE, K clone C38-2; BD Pharmingen, Heidelberg, Germany) were transferred to microtiter plates coated with 10 pg/ml anti-mouse-IgE rat monoclonal IgG (clone-PC 284; The Binding Site, Schwetzingen, Germany). As secondary antibody, 2.5 pg/ml of biotinylated monoclonal rat anti-mouse IgE (clone R35-118; BD Pharmingen) was used, followed by incubation with DB OptElA Reagent Set B (BD Pharmingen). Signal intensities of labeled total murine IgE were measured at 450 nm in a standard microwell ELISA reader and results reported in ng/ml based on a standard curve of purified IgE. The determination of further immunoglobulin isotypes levels was performed with a combined multiplexed assay system Meso Scale Discovery electrochemiluminescence assay (MSD, USA).

1.6) Determination of Allergic Response in Lavage

Flow cytometry was performed to quantify cell surface markers of BAL cells using the following monoclonal antibodies: anti-CD8a, anti-Lytic, anti-CD4, anti-CD62L, anti-CD3, anti-CD25, anti- GO, anti-CD19, anti-CD11 b, anti-CD11 c, and anti-MHC-II (Fuchs H, Gailus-Durner V, Adler T, Aguilar-Pimentel J A, Becker L, Calzada-Wack J, et al. Mouse phenotyping, Methods 2011; 53:120-35). Data were acquired using a LSRII flow cytometer (BD Bioscience) and further analyzed with FACSDiVa (BD Bioscience) and Flowjo V.7.2.2 (Tree star, Ashland, USA) software. Cytokines from BAL were analyzed by a multiplex measurement of proinflammatory cytokines from Meso Scale Discovery electrochemiluminescence assay (MSD, USA).

1.7) Cell Culture

For the T and B cell stimulation spleen cell from wild type mice were isolated and cells were cultivated in 200 μl/well in RPMI complete in cell culture plates (100,000 cells/well). For T cell stimulation total splenocytes were incubated 4 days with 1 ng/ml II-2 and 1 pg/ml anti-CD3 (Low) or 5 ng/ml II-2 and 10 pg/ml anti-CD3 (High) and three varying concentrations of Tofacitinib 50 ng/ml, 150 ng/ml and 500 ng/ml. For B cell stimulation cells were stimulated 8 days by incubation with 1 pg/ml anti-CD40 and 1L4 1 ng/ml (Low) and with 1 pg/ml anti-CD40 and 5 ng/ml (High) and three varying concentrations of Tofacitinib 50 ng/ml, 150 ng/ml and 500 ng/ml. The number of viable cells was determined by CellTiter-Glo Assay (Promega) and IgE concentration in the supernatant was measured by ELISA.

For the differentiation of human naive CD4⁺ T cells into FOXP3⁺ CD4⁺ T cells, human peripheral blood mononuclear cells were isolated from heparinized whole blood by standard density gradient centrifugation (Lymphoprep, Axis Shield, Oslo, Norway). Naive CD4⁺ T cells were isolated using the Naive CD4⁺ T cell Kit II (Miltenyi Biotec, Bergisch Gladbach, Germany) and by additional depletion of CD45RO⁺ cells (CD45RO microbeads, Miltenyi Biotec). Cells were cultured in RPMI 1640 (Thermo Fisher Scientific, Waltham, Mass. USA) complete (10% FCS, Biochrom, Merck, Berlin, Germany; 2 mM L-glutamine, 100 U/mL Penicillin/Streptomycin, Thermo Fisher Scientific) in 24-well plates at a concentration of 1×10⁶ cells/mL at 37° C. After isolation, cells were stimulated with plate-bound anti-CD3 (10 μg/mL; clone UCHT1, BD Biosciences) and 2 μg/mL anti-CD28 (clone CD28.2, BD Biosciences, 2 μg/mL) in solution and cultured for five days. To induce FOXP3 expression, the culture medium was supplemented with 50 U/mL rIL-2 (Novartis, Nurnberg, Germany) and 5 ng/mL TGF-01 (Promokine). Beneath this control, cells were cultured in the presence of different concentrations of Tofacitinib, Rapamycin (Sigma-Aldrich) and Cyclosporine A (Sigma-Aldrich). After three days of culture, half of the medium was removed and replaced by fresh RPMI 1640 complete, supplemented with the same doses of cytokines and agents as at day 0.

After five days of culture, the cells were washed with ice-cold PBS and stained with LIVE/DEAD Fixable Aqua Dead Cell Stain Kit (Thermo Fisher Scientific) according to the manufacturer's protocol. Moreover the cells were stained 1:300 with anti-CD4-Alexa Flour700 (BioLegend) and 1:100 with anti-FOXP3-eFlour450 (eBiosciences, affymetrix, Frankfurt am Main, Germany) by using the FOXP3/Transcription Factor Staining Buffer Set (eBiosciences, affymetrix) according to manufacturer's protocol. The acquisition of cells was performed with BD FACSDIVA 7.0 on a BD

LSR Fortessa (BD Biosciences). Data were analyzed with the software FlowJo (Tree Star, Ashland, Oreg.), the lymphocyte population was gated on CD4⁺ cells and dead cells were excluded from the analysis.

1.8) Statistical Analysis

If not otherwise indicated significant differences were calculated using the Mann-Whitney rank-sum test.

2) Results

2.1) Cell Culture Test

T Cell Stimulation

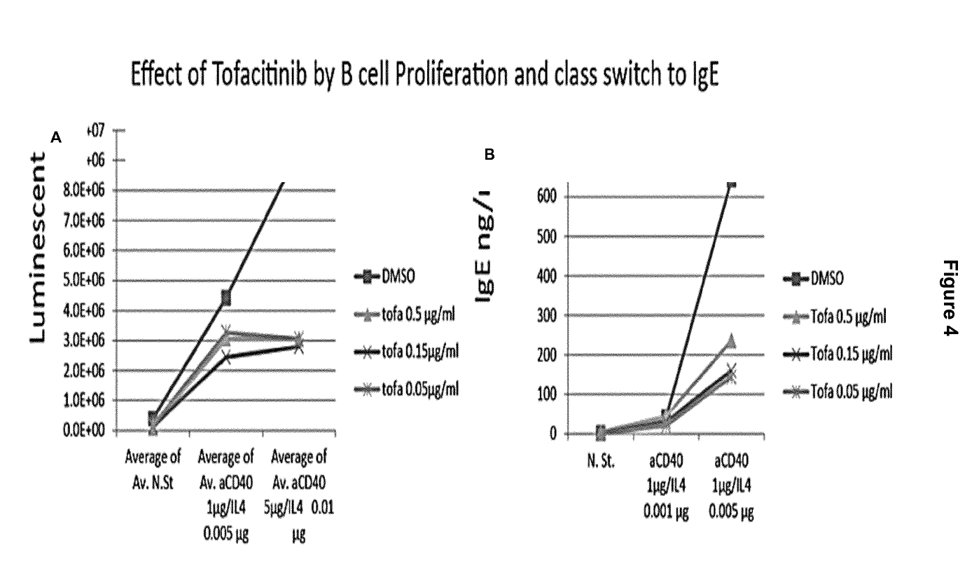

Under cell culture conditions T cells were stimulated by two different doses of activating stimulant. Tofacitinib significantly decreased proliferation of T cells in a dose-dependent manner (FIG. 3).

B Cell Stimulation

Under cell culture conditions B cells were stimulated by two different doses of activating stimulant. Tofacitinib significantly decreased proliferation of B cells (FIG. 4A). This effect was not as pronounced as observed for T cell proliferation. The treatment with Tofacitinib decreased the IgE production by B cells under cell culture conditions (FIG. 4B).

2.2) Tofacitinib Facilitated ASIT-Mediated Inhibition of BAL Cell Infiltration

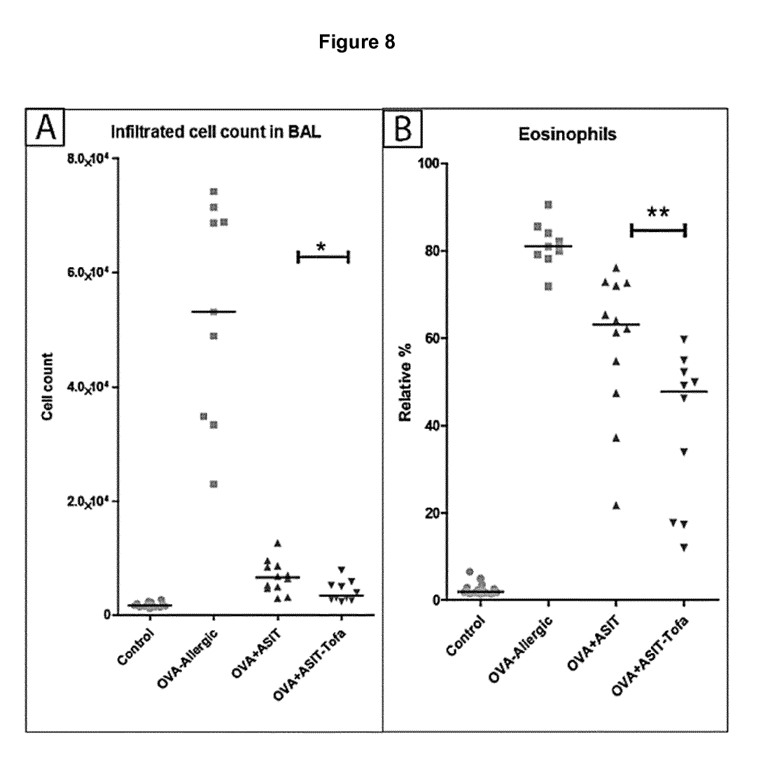

OVA-ASIT ameliorated OVA-induced total broncho-alveolar lavage cell infiltration in OVA-ASIT mice by 88% compared to OVA-allergic mice (cells events count 53162 [34141, 70157] Median [1st quantile; 3rd quantile]). The combination treatment using OVA+Tofacitinib further lead to further significant suppression (135 0.05) of the BAL infiltrate by 94% in OVA-TOFA-ASIT mice (FIG. 8A) compared to OVA-allergic mice.

Moreover, OVA-ASIT improved OVA-induced eosinophil BAL cell infiltration in OVA-SIT mice by 63.18% [49.29, 72.54] compared to OVA-allergic mice (81% [78.74, 84.88]). An additional significant (p5 0.005) reduction by 47.75% [17.63, 52.93] of eosinophil infiltration was achieved by combination treatment using OVA+Tofacitinib (FIG. 8B).

2.3) Tofacitinib Enhanced ASIT-Mediated Reduction of Total IgE

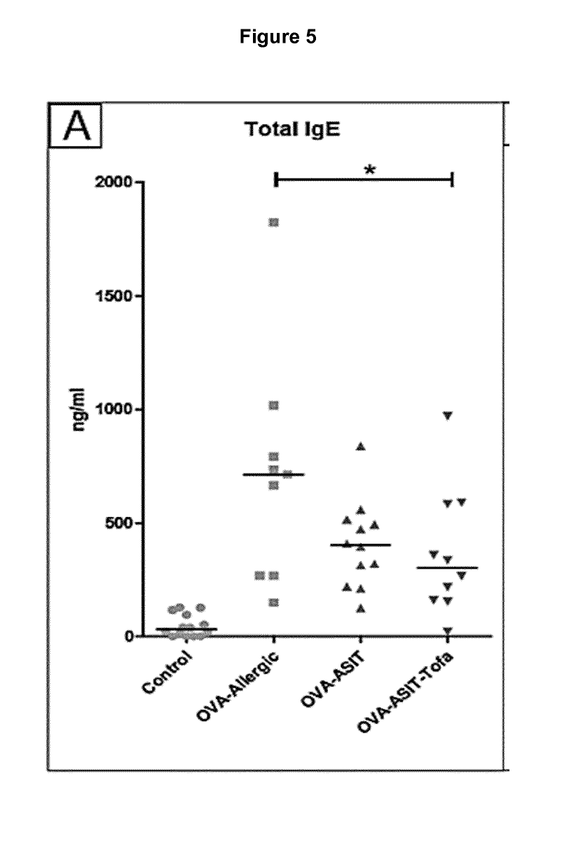

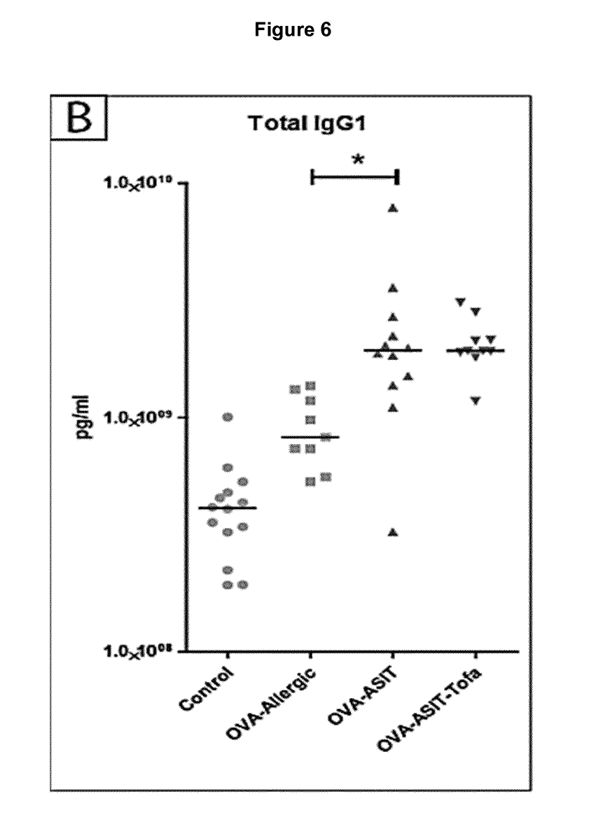

OVA-ASIT significantly (r) 0.05) enhanced OVA-induced total IgG1 levels in OVA-SIT mice to 1.936 mg/ml [1.408, 2.583] compared to OVA-allergic mice 0.8267 mg/ml [0.6485, 1.254]. The combination treatment using OVA+Tofacitinib had no impact on IgG1 levels (FIG. 6). In contrast, a tendency towards reduced total IgE-levels (43.5%) was observed for OVA-SIT, which was significant in case of the combination treatment OVA-TOFA-ASIT (57.8%) (FIG. 5).

2.4) Tofacitinib Enhanced ASIT-Mediated Reduction of Monokine and IL-4 Secretion in BAL Fluid

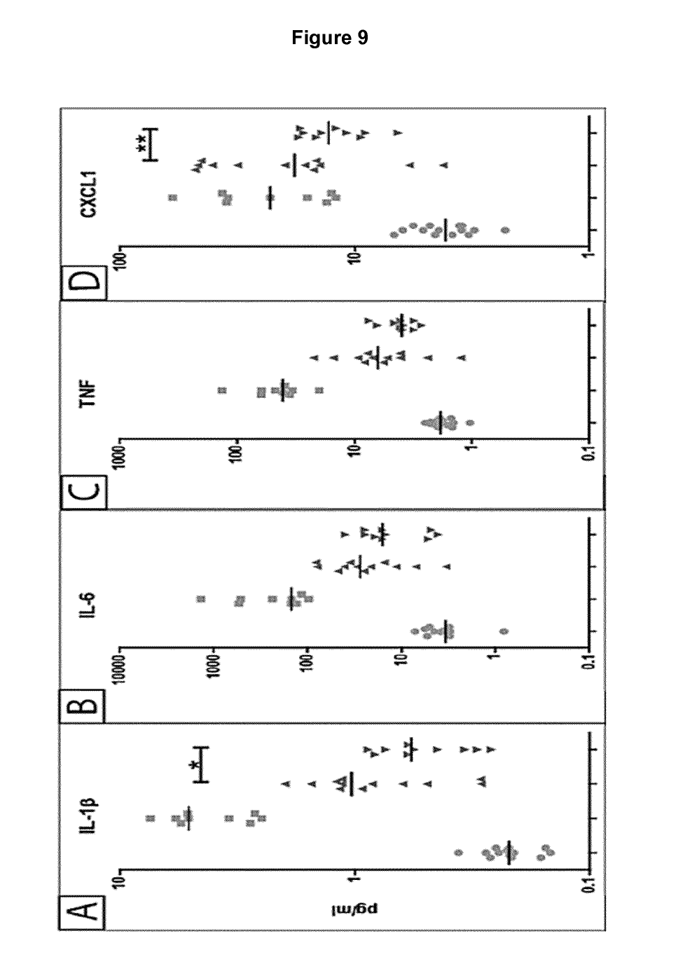

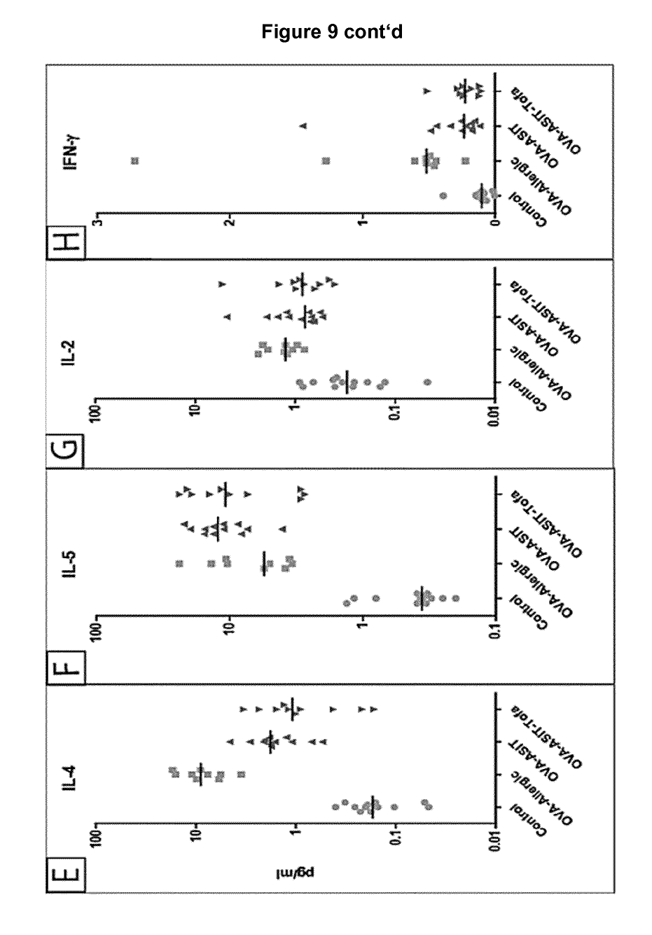

A dramatic inhibition of monokine secretion (IL-1 beta, IL-6 and TNF-a) in BAL fluid was induced by OVA-ASIT and OVA-TOFA-ASIT (80%, 82%, 84% and 89%, 89% and 90% respectively; FIG. 9 A-C). The addition of TOFA to OVA-ASIT most dramatically reduced (p5 0.05) IL-1 beta secretion compared to OVA-ASIT, while a tendency was observed for further reduction of IL-6 and TNF-a secretion. A similar enhancement of inhibition (21% and 44%) was observed for the chemokine CXCL1 (a murine homologue to human IL-8; FIG. 9 D). In contrast T cell cytokines IL-2 and IFN-γ were barely detectable and marginally reduced by OVA-ASIT without further effect by concomitant Tofacitinib treatment (FIG. 9 G-H).

While the secretion of the Th2-cytokine IL-4 was reduced in BAL by OVA-ASIT and a tendency towards further inhibition by OVA-TOFA-ASIT was observed, an increase of IL-5 secretion was observed under both treatment regimens (FIG. 9 E-F).

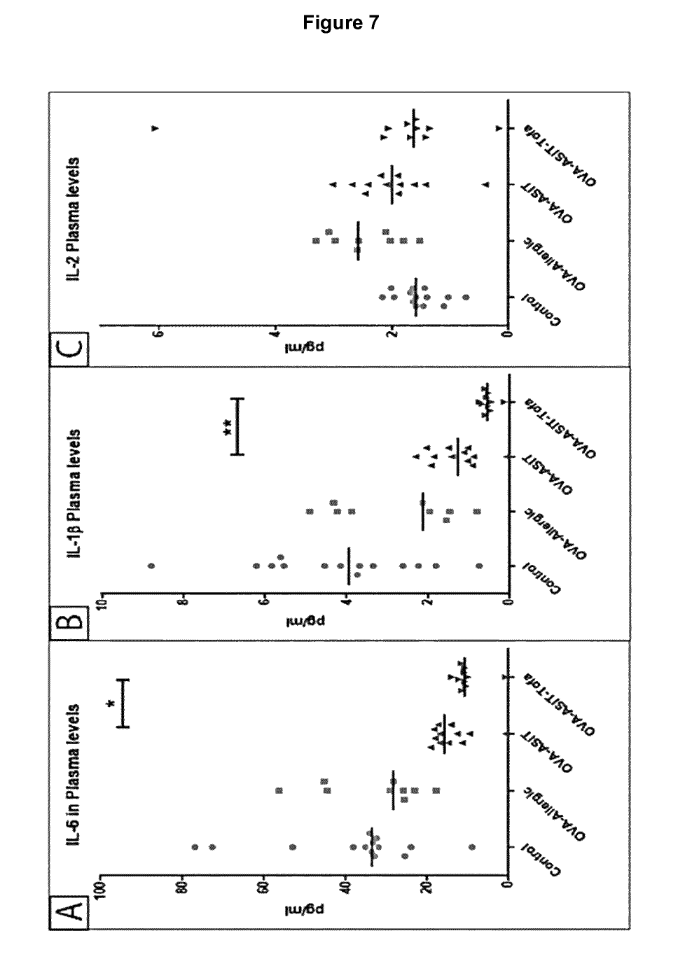

2.5) Tofacitinib Reinforced Reduction of Systemic IL-6—but not IL-2-Levels In contrast to elevated IL-6 levels in BAL-fluid, no systemic elevation was observed in OVA-allergic mice following OVA-aerosol inhalation. Interestingly, a reduction of IL-6 plasma levels (44%) was detected in OVA-ASIT mice with further significant reduction in OVA-TOFA-mice ((62%; p5 0.05; FIG. 7A). A similar enhancement of inhibition (41% and 75%) was observed for IL-lbeta (p5 0.05; FIG. 7B). In contrast, no significant effects on IL-2 plasma levels were exerted by OVA-ASIT or OVA-TOFA ASIT (FIG. 7C).

2.6) Tofacitinib Facilitates the Differentiation of Human FOXP3⁺ CD4⁺ T Cells

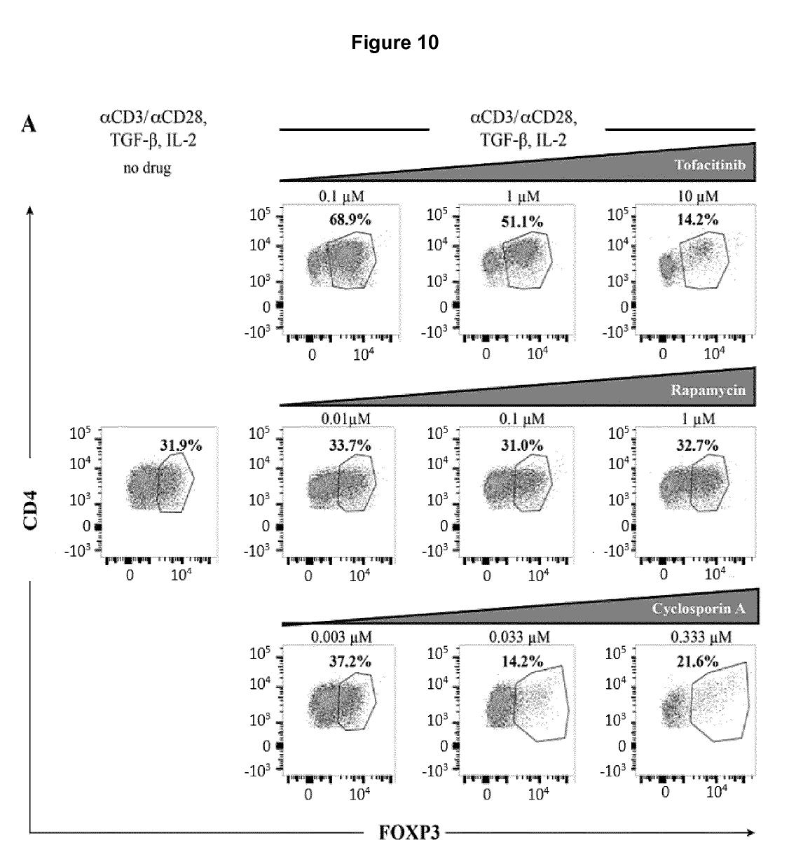

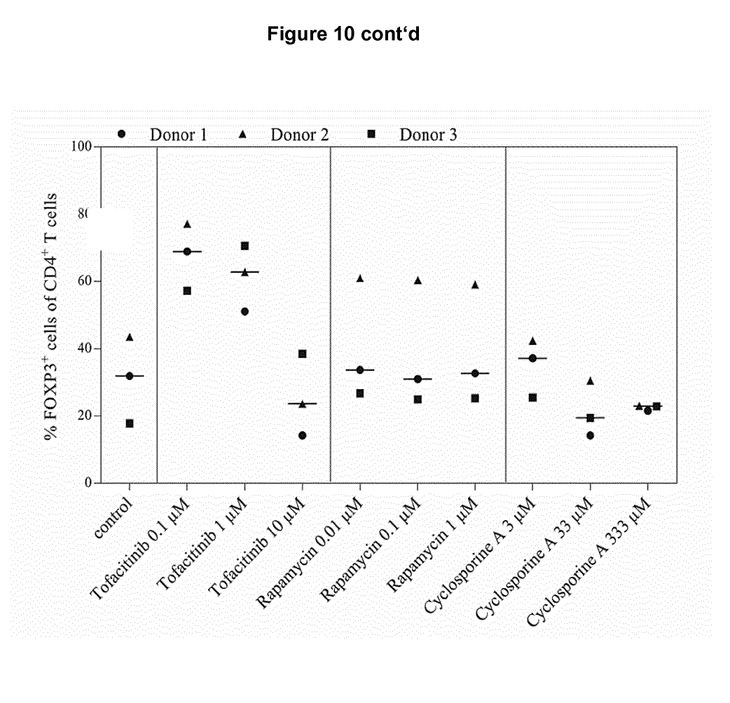

Since the suppressive effect of Tofacitinib on human and murine Th1/Th2/Th17 effector cells and immunoglobulin secretion has been shown previously, we analyzed the Treg-induction as important immune tolerance mechanism. In order to obtain insights into the effect of Tofacitinib and other immune-modulating agents such as Rapamycin and Cyclosporine A on FOXP3 expression in human CD4+ T cells, naive CD4+ T cells (purity 95-98%, data not shown) were isolated from healthy human blood donors (n=3) and cultured in the presence of TGF-131, IL-2 and different concentrations of the immune modulators. For the control cells, cultured under iTreg-polarizing conditions without immune modulators, 17.7-43.6% FOXP3⁺ cells within the living CD4⁺ T cell population were detected (FIG. 10). The two lower doses of Tofacitinib were able to enhance the induction of FOXP3 expression within this T cell compartment (FIG. 10).

For a concentration of 0.1 μM and 1 μM Tofacitinib in the culture medium 57.2-77.1% and 51.1-70.6% FOXP3 producing cells were detected, respectively. On average 0.1 μM and 1 μM Tofacitinib were able to increase the percentage of FOXP3 expressing cells about 2.4 (+/−0.7)-2.5 (+/−1.3)-fold. The concentration of 10 μM of Tofacitinib induced cytotoxicity (data not shown) and diminished the FOXP3-enhancing effect. Rapamycin treatment did not inhibit FOXP3 expression and resulted in 1.3-fold higher expression. The lowest concentration of Cyclosporine A (0.003 μM) showed no effect on FOXP3 induction, whereas the two higher concentrations (0.033 μM, 0.333 μM) reduced the percentage of FOXP3⁺ cells within the CD4⁺ T cell compartment. The obtained results were consistent for all individual donors (FIG. 10B).

3) Discussion

Several key parameters of experimental ASIT were improved by application of the JAK1/3 inhibitor Tofacitinib in combination with an established murine model of ASIT (Vissers J L, van Esch B C, Hofman G A, Kapsenberg M L, Weller F R, van Oosterhout A J. Allergen immunotherapy induces a suppressive memory response mediated by IL-10 in a mouse asthma model, J Allergy Clin Immunol 2004; 113:1204-10): Eosinophilic infiltration of the BAL-fluid, as well as BAL-levels of the Th2-cytokine IL-4, the monokines TNF-α, IL-6 and IL-1beta.

A key problem of human ASIT are the side effects, such as local swelling, local granuloma formation, anaphylaxis, as well as often insufficient clinical effect size, which are related to the local inflammatory conditions. The current study demonstrates that Tofacitinib-mediated JAK1/3 inhibition can significantly support ASIT-mediated control of local inflammation in particular the Th2 dependent influx of eosinophils.

Although the analysis of clinical trials is too multifactorial and requires large sample sizes, it can be reasonably anticipated that granulocyte and eosinophil reduction positively relate to clinical efficacy. The Tofacitinib-improved eosinophil infiltration is not related to Th2 cytokines, in particular IL-5, that was not decreased by OVA ASIT or OVA-Tofa ASIT. Interestingly IL-4 was differentially regulated and efficiently inhibited by OVA-Tofa-ASIT. Since IL-4 and IL-5 are under control of the same locus control region, it is unlikely that the differential secretion levels originate from the same cells, but rather are result of differential cell origin such as Th2 cells and innate lymphoid type 2 cells (Ikutani M, Yanagibashi T, Ogasawara M, Tsuneyama K, Yamamoto S, Hattori Y, et al. Identification of innate IL-5-producing cells and their role in lung eosinophil regulation and antitumor immunity, J Immunol 2012; 188:703-13).

The induction of IgG1 by OVA-ASIT and OVA-TOFA-ASIT is in line with previous studies, which have shown that OVA-ASIT in murine allergic airway disease induces OVA-specific IgG1 (Shirinbak S, Taher Y A, Maazi H, Gras R, van Esch B C, Henricks P A, et al. Suppression of Th2- driven airway inflammation by allergen immunotherapy is independent of B cell and Ig responses in mice, J Immunol 2010; 185:3857-65; Anderson C F, Gerber J S, Mosser D M. Modulating macrophage function with IgG immune complexes, J Endotoxin Res 2002; 8:477-81 and Sutterwala F S, Noel G J, Salgame P, Mosser D M. Reversal of proinflammatory responses by ligating the macrophage Fcgamma receptor type I, J Exp Med 1998; 188:217-22). Complexes of allergen-IgG can further induce Ig1 and the secretion of macrophage-derived IL-10, which is an important mediator of immune tolerance in experimental murine ASIT (Anderson C F, Gerber J S, Mosser D M. Modulating macrophage function with IgG immune complexes, J Endotoxin Res 2002; 8:477-81). These IgG1 antibodies are believed to compete with IgE in a similar way as IgG4 in the human system, which prevents mediator release from sensitized mast cells (Schmidt-Weber C B, Blaser K. Immunological mechanisms of specific allergen immunotherapy, Inflamm Allergy Drug Targets 2006; 5:15-21 and Wu Z. Antigen specific immunotherapy generates CD27(+) CD35(+) tolerogenic dendritic cells, Cell Immunol 2013; 283:75-80). However, the reduction of Th2-late phase response with eosinophil infiltration in the BAL-fluid is independent from B cell function and IgG-production (Shirinbak S, Taher Y A, Maazi H, Gras R, van Esch B C, Henricks P A, et al. Suppression of Th2-driven airway inflammation by allergen immunotherapy is independent of B cell and Ig responses in mice, J Immunol 2010; 185:3857-65).

The effect of Tofacitinib-enhanced ASIT was most pronounced among monokines IL-1beta, TNF-α and IL-6, despite the fact that the last application of Tofacitinib was given 18 days prior to allergen challenge. This finding is surprising since the mean terminal plasma half-life of Tofacitinib is 3.2 hours (Dowty M E, Lin J, Ryder T F, Wang W, Walker G S, Vaz A, et al. The pharmacokinetics, metabolism, and clearance mechanisms of tofacitinib, a janus kinase inhibitor, in humans, Drug Metab Dispos 2014; 42:759-73). Moreover, it has been shown that discontinuation of Tofacitinib after short-term treatment in vitro leads to reactivation of lymphocytes within four days (Piscianz E, Valencic E, Cuzzoni E, De ludicibus S, De Lorenzo E, Decorti G, et al. Fate of lymphocytes after withdrawal of tofacitinib treatment, PLoS One 2014; 9:e85463). However, the reduction of IL-6 has also been observed in a four weeks clinical trial assessing Tofacitinib for rheumatoid arthritis (Migita K, Izumi Y, Jiuchi Y, Kozuru H, Kawahara C, Izumi M, et al. Effects of Janus kinase inhibitor tofacitinib on circulating serum amyloid A and interleukin-6 during treatment for rheumatoid arthritis, Clin Exp Immunol 2014; 175:208-14). Taken together, it is likely that resident epithelial cells (e.g. bronchial/alveolar or liver epithelial cells) mediate the reduction of proinflammatory mediators, as it has been shown in a recent model of graft-versus host disease for keratinocyte-derived CXCL9 and CXCL10 (Okiyama N, Furumoto Y, Villarroel V A, Linton J T, Tsai W L, Gutermuth J, et al. Reversal of CD8 T-cell-mediated mucocutaneous graft-versus-host-like disease by the JAK inhibitor tofacitinib, J Invest Dermatol 2014; 134:992-1000).

Taken together, the combination treatment of ASIT plus Tofacitinib seems to amplify the clinical efficacy of ASIT, as it not only affects adaptive, but also the innate arm of immune responses.

Additionally, Tofacitinib administration in vitro favored the induction of human FOXP3⁺ CD4⁺ T cells, a hallmark of T cell tolerance (Akdis M and Akdis C A (2014) Mechanisms of allergen-specific immunotherapy: multiple suppressor factors at work in immune tolerance to allergens. J Allergy Clin Immunol 133:621-31). This extends the findings of an earlier study, which demonstrated that Tofacitinib preserves the function of regulatory T cells and inhibits effector T cells (Sewgobind V D, Quaedackers M E, van der Laan L J et al. (2010) The Jak inhibitor CP-690,550 preserves the function of CD4CD25FoxP3 regulatory T cells and inhibits effector T cells. Am J Transplant 10:1785-95) In contrast, other immunosuppressive drugs such as Cyclosporine A inhibit FOXP3-mediated Treg induction (Miroux C, Morales O, Carpentier A et al. (2009) Inhibitory effects of cyclosporine on human regulatory T cells in vitro. Transplant Proc 41:3371-4). We previously demonstrated that this effect depends on direct inhibition of NFAT binding to the proximal FOXP3 promoter (Mantel P Y, Ouaked N, Ruckert B et al. (2006) Molecular mechanisms underlying FOXP3 induction in human T cells. J Immunol 176:3593-602). Our data demonstrate that JAK-inhibitors are able to shift the balance of newly induced T cell reactivity. This potentially favors the induction of functional regulatory T cells, which build the basis for a long-lasting modulation of the immunological memory and which are beneficial for the treatment of a variety of immune diseases.

1. A pharmaceutical composition for use in medicine, wherein said composition comprises Tofacitinib or a pharmaceutically acceptable salt thereof, and at least one antigen or allergen.

2. The pharmaceutical composition for use according to claim 1, further comprising at least one adjuvant, preferably wherein the adjuvant is selected from monophosphoryl lipid A (MPL), alum or activators of dendritic cells.

3. The pharmaceutical composition for use according to any one of claims 1 and 2, further comprising at least one pharmaceutically acceptable carrier.

4. The pharmaceutical composition for use according to any one of claims 1-3, wherein the antigen is an autoantigen, preferably wherein the autoantigen is selected from DNA components, pancreatic islet antigens, GAD, insulinoma-associated antigen 2, insulin, zinc transporter 8, RPS, Alba-like, p62, CYPs, dUTPase, ASGPR2, SMA, matrix antigens, citrullin, carbamylated protein, liver and kidney microsomal antigens, Asialoglycoprotein receptor, neutrophil related antigens, extracellular matrix-proteins, MHC-II eluted peptides.

5. The pharmaceutical composition for use according to any one of claims 1-4, wherein Tofacitinib or a pharmaceutically acceptable salt thereof is included in a pharmaceutically acceptable carrier that also contains the antigen or allergen and optionally at least one adjuvant and/or further excipient.

6. A pharmaceutical composition for use as a tolerogenic vaccine in the treatment or prevention of an immune disease, wherein said composition comprises Tofacitinib or a pharmaceutically acceptable salt thereof and at least one antigen or allergen.

7. The pharmaceutical composition for use according to claims 6, further comprising at least one adjuvant.

8. The pharmaceutical composition for use according to any one of claims 6-7, further comprising at least one pharmaceutically acceptable carrier.

9. The pharmaceutical composition for use according to any one of claims 6-8, wherein the immune disease is an allergy or asthma.

10. The pharmaceutical composition for use according to claim 9, wherein the allergy is selected from allergic rhinitis, hay fever, allergic airway inflammation and food allergies, preferably allergic rhinitis.

11. The pharmaceutical composition for use according to claim 9, wherein the asthma is selected from allergic asthma or asthma with unknown triggers (autoimmune, autoallergies).

12. The pharmaceutical composition for use according to any one of claims 6-11, wherein the immune disease is an autoimmune disease.

13. The pharmaceutical composition for use according to claim 12, wherein the autoimmune disease is selected from Type I diabetes, multiple sclerosis, ulcerative colitis, Crohn's disease, lupus erythematosus, psoriasis, rheumatoid arthritis, psoriatic arthritis, GravesDisease, Hashimoto's Thyroiditis, Vitiligo, Pernicious Anemia, Scleroderma, Glomerulonephritis, Sjogren's Syndrome, Bullous pemphigoid and Pemphigus vulgaris, preferably wherein the disease is Type I diabetes and/or multiple sclerosis, GravesDisease, Hashimoto's Thyroiditis, Vitiligo, Pernicious Anemia, Glomerulonephritis, ulcerative colitis, Crohn's disease, Sjogren's Syndrome, Bullous pemphigoid, Scleroderma and Pemphigus vulgaris, more preferably ulcerative colitis, Crohn's disease, Pemphigus vulgaris, Bullous Pemphigoid and Scleroderma.

14. The pharmaceutical composition for use according to any one of claims 6-13, wherein said composition is administered to a subject in need thereof in an amount effective to treat or prevent said immune diseases, preferably wherein the subject is a human subject.

15. Tofacitinib or a pharmaceutically acceptable salt thereof for use in tolerogenic vaccination.

16. Tofacitinib or a pharmaceutically acceptable salt thereof for use according to claim 15, wherein said compound supports a tolerogenic vaccination.

17. Tofacitinib or a pharmaceutically acceptable salt thereof for use according to any one of claims 15-16, wherein said compound is administered orally shortly before or simultaneously with an antigen or allergen vaccination, preferably wherein the used vaccine contains at least one adjuvant.

18. Tofacitinib or a pharmaceutically acceptable salt thereof for use according to any one of claims 15-17, wherein said compound is an immune modulator, in particular a tolerance inducer.

19. Tofacitinib or a pharmaceutically acceptable salt thereof for use according to claims 15-18, wherein said compound promotes the induction of the allergen or antigen tolerance.

20. Tofacitinib or a pharmaceutically acceptable salt thereof for use in the treatment or prevention of allergic rhinitis.

21. Tofacitinib or a pharmaceutically acceptable salt thereof for use according to claim 20, wherein said compound is administered orally shortly before or simultaneously with an allergen vaccination for allergic rhinitis.

22. Tofacitinib or a pharmaceutically acceptable salt thereof for use in the treatment or prevention of an immune disease, wherein said compound is administered orally shortly before or simultaneously with an antigen vaccination, preferably wherein the immune disease is an autoimmune disease.

23. Kit of parts comprising Tofacitinib or a pharmaceutically acceptable salt thereof, and at least one antigen or allergen.

24. Kit of parts according to claims 23, further comprising at least one adjuvant and/or pharmaceutically acceptable carrier.

25. Kit of parts according to claim 23, wherein said compound and the allergen or antigen are included in a pharmaceutically acceptable carrier, optionally with at least one adjuvant and/or further excipient.
